(19) United States
(12) Patent Application Publication
(10) Pub. No.: US 2020/0121277 A1
(43) Pub. Date: Apr. 23, 2020

(54) SYSTEMS AND METHODS FOR DETECTING PHYSIOLOGICAL INFORMATION USING A SMART STETHOSCOPE

(71) Applicant: Deep Science, LLC, Bellevue, WA (US)

(72) Inventors: Roderick A. Hyde, Redmond, WA (US); David William Wine, Seattle, WA (US); Mary Neuman, Seattle, WA (US); Roger Zundel, Bellevue, WA (US); Brian C. Holloway, Snoqualmie, WA (US)

(21) Appl. No.: 16/657,596

(22) Filed: Oct. 18, 2019

Related U.S. Application Data
(60) Provisional application No. 62/747,617, filed on Oct. 18, 2018.

(51) Int. Cl. A61B 7/04; A61B 5/021; H04R 3/04; G06N 20/00

(52) U.S. Cl. CPC A61B 7/04; A61B 5/021; H04R 2499/11; G06N 20/00; H04R 2420/07; H04R 3/04

(57) ABSTRACT

A stethoscope system includes a microphone device configured to receive a plurality of sound waves from the subject and output an audio signal corresponding to the plurality of sound waves; and a control circuit configured to receive the audio signal from the microphone device and calculate a physiological parameter based on the audio signal.

### CROSS-REFERENCE TO RELATED APPLICATIONS

The present disclosure claims the benefit of and priority to U.S. Provisional Application No. 62/747,617, titled “SYSTEMS AND METHODS OF MICRO IMPULSE RADAR DETECTION OF PHYSIOLOGICAL INFORMATION,” filed Oct. 18, 2018, the disclosure of which is incorporated herein by reference in its entirety.

### BACKGROUND

The present disclosure relates generally to the field of diagnostic sensors. More particularly, the present disclosure relates to systems and methods for detecting physiological information using an electronic stethoscope.

Stethoscopes can be used to receive audio information from a subject. For example, stethoscopes can be used to monitor audio from lungs or the heart of the subject.

### SUMMARY

At least one embodiment relates to a stethoscope system. The system includes a microphone device configured to receive a plurality of sound waves from the subject and output an audio signal corresponding to the plurality of sound waves; and a control circuit configured to receive the audio signal from the microphone device and calculate a physiological parameter based on the audio signal.

Another embodiment relates to a method. The method includes receiving, by a microphone device, a plurality of sound waves from a subject; outputting, by the microphone device, an audio signal corresponding to the plurality of sound waves; and calculating, by a control circuit, a physiological parameter based on the audio signal.

This summary is illustrative only and is not intended to be in any way limiting.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a block diagram of a stethoscope device in accordance with an embodiment of the present disclosure.

FIG. 2 is a block diagram of a stethoscope system in accordance with an embodiment of the present disclosure.

FIG. 3 is a flow diagram of a method of operating a stethoscope system in accordance with an embodiment of the present disclosure.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

### A. Systems and Methods for Detecting Physiological Parameters Using an Electronic Stethoscope

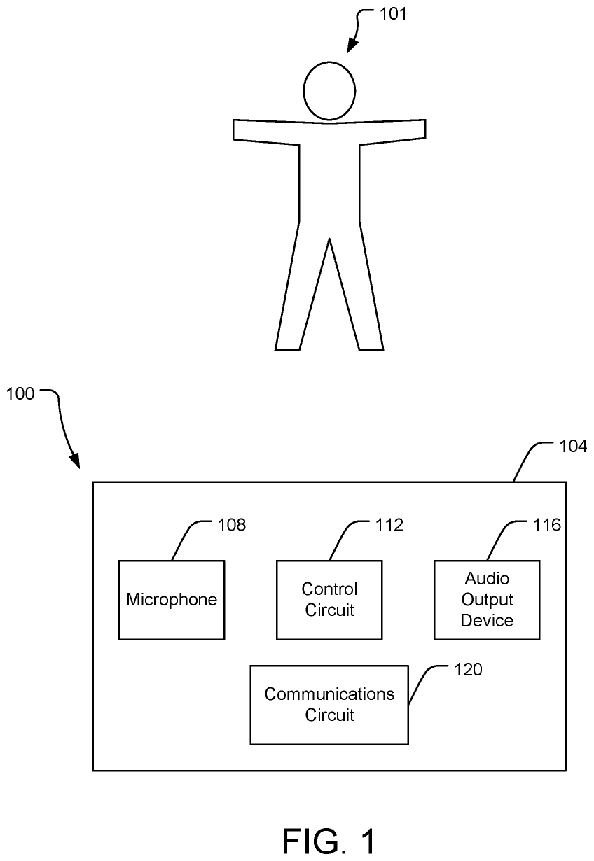

Referring now to FIG. 1, a medical device (e.g., a stethoscope device) 100 is shown according to an embodiment of the present disclosure. The stethoscope device 100 includes a housing 104 supporting a microphone 108, a control circuit 112, and an audio output device 116.

The housing 104 can be sized to be hand-held to enable the stethoscope device 100 to be manipulated around the subject 101. In some embodiments, the housing 104 is wearable. As such, the stethoscope device 100 can be worn for relatively long durations of time, enabling the stethoscope device 100 to receive and provide for storage much greater durations of audio information than existing stethoscope systems, and thus enabling longitudinal studies.

The microphone 108 can receive sound waves and output an electronic audio signal corresponding to the sound waves. For example, the microphone 108 can be positioned in proximity to a sound source (e.g., the subject 101) to receive the sound waves from the sound source. The microphone 108 can be positioned to receive sound waves from the heart, lungs, abdominal cavity, or other portions of the subject 101.

The control circuit 112 can include a processor and memory. The processor may be implemented as a specific purpose processor, an application specific integrated circuit (ASIC), one or more field programmable gate arrays (FPGAs), a system on a chip (SoC), a group of processing components (e.g., multicore processor), or other suitable electronic processing components. The memory 316 is one or more devices (e.g., RAM, ROM, flash memory, hard disk storage) for storing data and computer code for completing and facilitating the various user or client processes, layers, and modules described in the present disclosure. The memory may be or include volatile memory or non-volatile memory and may include database components, object code components, script components, or any other type of information structure for supporting the various activities and information structures of the inventive concepts disclosed herein. The memory is communicably connected to the processor and includes computer code or instruction modules for executing one or more processes described herein. The memory includes various circuits, software engines, and/or modules that cause the processor to execute the systems and methods described herein.

The control circuit 112 can process the electronic audio signal to generate an output audio signal for output via the audio output device 116. For example, the control circuit 112 can amplify, filter, attenuate, or otherwise modify the electronic audio signal. The audio output device 116 can include a speaker to output the audio output device 116 as output sound waves to be heard by a user.

In some embodiments, the control circuit 112 provides the electronic audio signal (processed or unprocessed) to a communications circuit 120. 1 The communications circuit 120 can transmit the electronic audio signal to a remote device for further processing. The communications circuit 120 can include wired or wireless interfaces (e.g., jacks, antennas, transmitters, receivers, transceivers, wire terminals, etc.) for conducting data communications with various systems, devices, or networks. For example, the communications circuit 120 can include an Ethernet card and port for sending and receiving data via an Ethernet-based communications network. The communications circuit 120 can include a WiFi transceiver for communicating via a wireless communications network. The communications circuit 120 can communicate via local area networks (e.g., a building LAN), wide area networks (e.g., the Internet, a cellular network), and/or conduct direct communications (e.g., NFC, Bluetooth). In some embodiments, the communications circuit 120 can conduct wired and/or wireless communications. For example, the communications circuit 120 can include one or more wireless transceivers (e.g., a Wi-Fi transceiver, a Bluetooth transceiver, a NFC transceiver, a cellular transceiver).

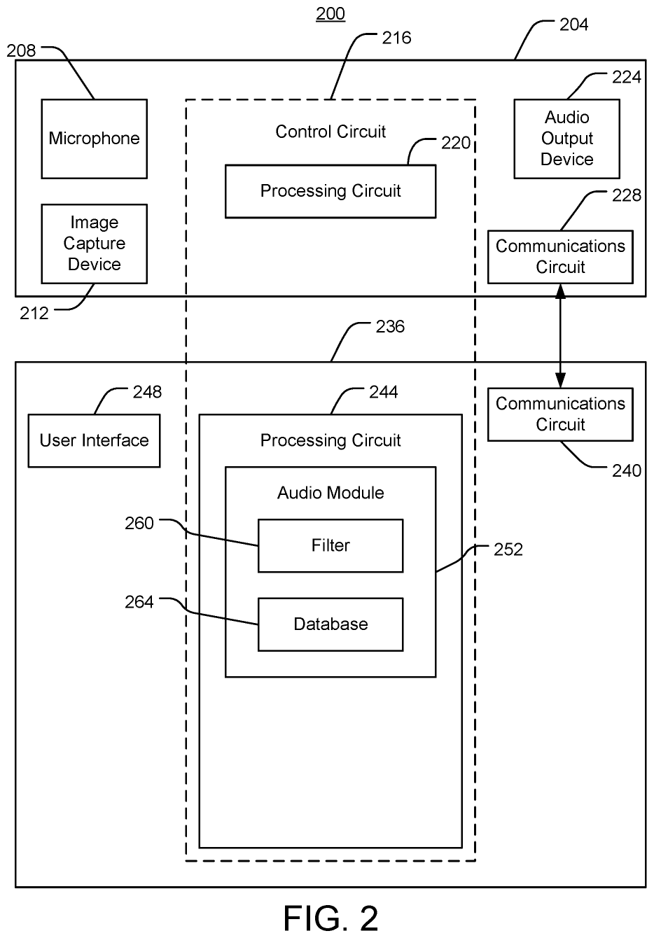

Referring now to FIG. 2, a medical device system (e.g., a stethoscope system) 200 is shown according to an embodiment of the present disclosure. The stethoscope system 200 can incorporate features of the stethoscope device 100 described with reference to FIG. 1.

As shown in FIG. 2, the stethoscope system 200 includes a stethoscope device 204 including a microphone 208, a control circuit 216 including a processing circuit 220, an audio output device 224, and a communications circuit 228. The processing circuit 220 can receive an electronic audio signal from the microphone 208, and provide an audio output signal based on the electronic audio signal to the audio output device 224 and/or the communications circuit 228.

The stethoscope system 200 includes a remote stethoscope unit 236 that can enable the stethoscope system 200 to perform additional functionality without increasing processing power requirements, size, weight, power, and/or cost of the stethoscope device 204. It will appreciated that functionality described with respect to the remote stethoscope unit 236 may be performed by a portable electronic device (e.g., cell phone), a cloud-based server in communication with the remote stethoscope unit 236 and/or the stethoscope device 204, or various combinations thereof based on such factors. For example, while FIG. 2 illustrates the filter 260 as being implemented by processing circuit 244 of remote stethoscope unit 236, the filter 260 (or functions thereof) can be implemented by processing circuit 220.

The remote stethoscope unit 236 includes a processing circuit 244 and a communications circuit 240. The processing circuit 244 can cooperate with the processing circuit 220 to perform the functions of the control circuit 216 described herein, including by communicating with the processing circuit 220 using the communications circuits 228, 240.

The control circuit 216 includes an audio module 252. The audio module 252 can include a parameter calculator, a historical database, a health condition calculator, and a machine learning engine.

The remote stethoscope unit 236 can include a user interface 248. The user interface 248 can receive user input and present information regarding operation of the stethoscope system 200. The user interface 248 may include one or more user input devices, such as buttons, dials, sliders, or keys, to receive input from a user. The user interface 248 may include one or more display devices (e.g., OLED, LED, LCD, CRT displays), speakers, tactile feedback devices, or other output devices to provide information to a user.

### Audio Processing and Analysis Module

The audio module 252 includes a filter 260 and an audio database 264. The filter 260 can execute various audio filters on the electronic audio signal received from the microphone 208. For example, the filter 260 can execute low-pass, high-pass, band-pass, notch, or various other filters and combinations thereof.

In some embodiments, the filter 260 executes one or more audio filters based on an expected physiological parameter represented by the electronic audio signal. For example, the audio database 264 may maintain a plurality of audio filter profiles, each audio filter profile corresponding to a respective type of physiological parameter. The filter 260 can receive an indication of the type of physiological parameter and retrieve the corresponding audio filter profile accordingly to generate a filter to apply to the electronic audio signal. For example, each audio filter profile may indicate a particular frequency range of interest for the physiological parameter. The audio filter profile may indicate various signal processing actions to apply to the electronic audio signal, including amplification and attenuation.

The audio module 252 can 2 determine physiological parameters and likelihoods of medical conditions based on the electronic audio signals. For example, the audio module 252 can determine physiological parameters based on the filtered electronic audio signals. The control circuit 216 can store the electronic audio signal or features thereof as a signature of the subject 101, which can later be retrieved to identify the subject 101 based on detecting a subsequent electronic audio signal of the subject 101.

The control circuit 216 can maintain, in the audio database 264, various subject parameter profiles. For example, a subject parameter profile may include an identifier of the subject, each electronic audio signal received for the subject, historical data regarding the subject, physiological parameters calculated for the subject, and likelihoods of medical conditions calculated for the subject. The audio database 264 can maintain data that can be used as a teaching tool (e.g., for educational or training purposes). For example, the control circuit 216 can receive a request to retrieve an electronic audio signal based on various request inputs (e.g., request for audio signals associated with a particular subject, with particular physiological parameters, or with particular medical conditions), search the audio database 264 using the request, and retrieve the corresponding electronic audio signals. The control circuit 216 can output the electronic audio signal along with characteristic information regarding the subject (e.g., age, sex, height, weight), physiological parameters associated with the subject, medical conditions associated with the subject, or various combinations thereof. As such, a user can review any number of electronic audio signals after the signals have been recorded to learn features of the signals and the relationships between the signals and various physiological parameters and medical conditions.

The control circuit 216 can execute a machine learning engine similar to machine learning engine 420 described with reference to FIG. 4 to generate and improve the accuracy of models used for calculating parameters based on the electronic audio signals. The control circuit 216 can combine the data of the audio database 264 with training data of other modalities to generate multi-modal models, which can have improved accuracy and predictive ability.

As shown in FIG. 2, the stethoscope system 200 also can include an image capture device 212. The image capture device 212 can capture images regarding the subject 101, and provide the images to the processing circuit 220 (e.g., to a historical database maintained by the processing circuit 220).

The processing circuit 220 can execute object recognition and/or location estimation using the images captured by the image capture device 212. For example, the processing circuit 312 can extract, from a received image, features such as shapes, colors, edges, and/or spatial relationships between pixels of the received images. The processing circuit 220 can compare the extracted features to template features (e.g., a template of a human subject), and recognize objects of the images based on the comparison, such as by determining a result of the comparison to satisfy a match condition. The template can include an expected shape of the subject 101. In some embodiments, the processing circuit 220 can estimate the location of anatomical features of the subject 101 based on the receive image, such as by estimating a location of a heart, lungs, or womb of the subject 101 based on having detected the subject 101.

### Parameter Calculator

The audio module 252 can use a parameter calculator to determine, based on the electronic audio signal, a physiological parameter of the subject. For example, the parameter calculator can calculate parameters such as locations of anatomical features, movement of anatomical features, movement of fluids (e.g., blood flow), or velocity data. The parameter calculator can calculate the physiological parameter to include at least one of a cardiac parameter, a pulmonary parameter, a blood flow parameter, or a fetal parameter based on the electronic audio signals.

In some embodiments, the parameter calculator calculates the physiological parameter using at least one of a predetermined template or a parameter function. The predetermined template may include features such as expected signal amplitudes at certain frequencies, or pulse shapes of the electronic audio signal.

In some embodiments, the parameter calculator calculates the physiological parameter based on an indication of a type of the physiological parameter. For example, the parameter calculator can receive the indication based on user input. The parameter calculator can determine the indication, such as by determining an expected anatomical feature of the subject 101 that the stethoscope system 200 is monitoring. For example, the parameter calculator can use image data from image capture device 212 to determine that the stethoscope system 200 is monitoring a heart of the subject 101, and determine the type of the physiological parameter to be a cardiac parameter. The parameter calculator may use the determined type of the physiological parameter to select a particular predetermined template or parameter function to execute, or to increase a confidence that the electronic audio signal represents the type of physiological parameter (which may be useful for calculating the physiological parameter based on comparing the electronic audio signal to predetermined template(s) and searching for a match accordingly).

### Historical Database

The audio database 264 can include a historical database that maintains historical data regarding a plurality of subjects, electronic audio signals received for each subject, physiological parameters calculated for each subject, and stethoscope system operations corresponding to the physiological parameters calculated for each subject. The historical database can maintain indications of intended physiological features to be monitored using the stethoscope system 200 (e.g., heart, lungs) and/or types of the calculated physiological parameters (e.g., cardiac, pulmonary). The historical database can assign to each subject various demographic data (e.g., age, sex, height, weight).

The historical database can maintain various parameters calculated based on electronic audio signals. For example, the historical database can maintain physiological parameters, signal to noise ratios, health conditions, and other parameters described herein that the processing circuits 220, 244 calculate using the electronic audio signals. The historical database can be updated when additional electronic audio signals are received and analyzed.

### Health Condition Calculator

In some embodiments, the audio module 252 implements a health condition calculator. The health condition calculator can use the physiological parameters calculated by the parameter calculator and/or the historical data maintained by the historical database to calculate a likelihood of the subject having a particular health condition. The health condition calculator 416 can calculate likelihoods associated with medical conditions, emotion conditions, physiological conditions, or other health conditions.

In some embodiments, the health condition calculator predicts a likelihood of the subject 101 having the health condition by comparing the physiological parameter to at least one of (i) historical values of the physiological parameter associated with the subject (e.g., as maintained in the historical database) or (ii) a predetermined value of the physiological parameter associated with the medical condition (e.g., a predetermined value corresponding to a match condition as described below). For example, the health condition calculator can calculate an average value over time of the physiological parameter to determine a normal value or range of values for the subject 101, and determine the likelihood of the subject 101 having the medical condition based on a difference between the physiological parameter and the average value.

The health condition calculator can maintain a match condition associated with each health condition. The match condition can include one or more thresholds indicative of radar return data and/or physiological parameters that match the health condition. The health condition calculator can store the outputted likelihoods in the historical database.

In some embodiments, the health condition calculator updates the match conditions based on external input. For example, the health condition calculator can receive a user input indicating a health condition that the subject 101 has; the user input may also include an indication of a confidence level regarding the health condition. The health condition calculator can adjust the match condition, such as by adjusting the one or more thresholds of the match condition, so that the match condition more accurately represents the information of the external input. In some embodiments, the health condition calculator updates the match condition by providing the external input as training data to a machine learning engine.

The health condition calculator can determine the likelihood of the subject 101 having the medical condition based on data regarding a plurality of subjects. For example, the historical database can maintain electronic audio data, physiological parameter data, and medical conditional data regarding a plurality of subjects (which the machine learning engine can use to generate richer and more accurate parameter models). The health condition calculator can calculate a statistical measure of a physiological parameter (e.g., average value, median value) for the plurality of subjects, and calculate an indication of the physiological parameter of the subject 101 being abnormal and/or calculate a likelihood of the subject 101 having the medical condition based on the statistical measure.

### Machine Learning Engine

In some embodiments, the audio module 252 includes a machine learning engine. The machine learning engine can be used to calculate various parameters described herein, including where relatively large amounts of data may need to be analyzed to calculate parameters as well as the thresholds used to evaluate those parameters. For example, the parameter calculator can execute the machine learning engine to determine the thresholds used to recognize physiological parameters. The medical condition calculator can execute the machine learning engine to determine the thresholds used to determine whether physiological parameters indicate that the subject 101 has a particular medical condition.

In some embodiments, the machine learning engine includes a parameter model. The machine learning engine can use training data including input data and corresponding output parameters to train the parameter model by providing the input data as an input to the parameter model, causing the parameter model to calculate a model output based on the input data, comparing the model output to the output parameters of the training data, and modifying the parameter model to reduce a difference between the model output and the output parameters of the training data (e.g., until the difference is less than a nominal threshold). For example, the machine learning engine can execute an objective function (e.g., cost function) based on the model output and the output parameters of the training data.

The parameter model can include various machine learning models that the machine learning engine can train using training data and/or the historical database. The machine learning engine can execute supervised learning to train the parameter model. In some embodiments, the parameter model includes a classification model. In some embodiments, the parameter model includes a regression model. In some embodiments, the parameter model includes a support vector machine (SVM). In some embodiments, the parameter model includes a Markov decision process engine.

In some embodiments, the parameter model includes a neural network. The neural network can include a plurality of layers each including one or more nodes (e.g., neurons, perceptrons), such as a first layer (e.g., an input layer), a second layer (e.g., an output layer), and one or more hidden layers. The neural network can include characteristics such weights and biases associated with computations that can be performed between nodes of layers, which the machine learning engine can modify to train the neural network. In some embodiments, the neural network includes a convolutional neural network (CNN). The machine learning engine can provide the input from the training data and/or historical database in an image-based format (e.g., computed radar values mapped in spatial dimensions), which can improve performance of the CNN as compared to existing systems, such as by reducing computational requirements for achieving desired accuracy in calculating health conditions. The CNN can include one or more convolution layers, which can execute a convolution on values received from nodes of a preceding layer, such as to locally filter the values received from the nodes of the preceding layer. The CNN can include one or more pooling layers, which can be used to reduce a spatial size of the values received from the nodes of the preceding layer, such as by implementing a max pooling function, an average pooling function, or other pooling functions. The CNN can include one or more pooling layers between convolution layers. The CNN can include one or more fully connected layers, which may be similar to layers of neural networks by connecting every node in fully connected layer to every node in the preceding layer (as compared to nodes of the convolution layer(s), which are connected to less than all of the nodes of the preceding layer).

The machine learning engine can train the parameter model by providing input from the training data and/or historical database as an input to the parameter model, causing the parameter model to generate model output using the input, modifying a characteristic of the parameter model using an objective function (e.g., loss function), such as to reduce a difference between the model output and the and the corresponding output of the training data. In some embodiments, the machine learning engine executes an optimization algorithm that can modify characteristics of the parameter model, such as weights or biases of the parameter model, to reduce the difference. The machine learning engine can execute the optimization algorithm until a convergence condition is achieved (e.g., a number of optimization iterations is completed; the difference is reduced to be less than a threshold difference).

### Audio Information Presentation

By maintaining electronic audio signals in the audio database 264, the control circuit 216 can enable audio manipulation and analysis not possible with typical stethoscope systems. For example, the control circuit 216 can use the user interface 248 to output visual and/or audio representations of electronic audio signals at various speeds. The control circuit 216 can highlight particular features of interest in the electronic audio signals. As compared to existing systems that rely on a user to subjectively evaluate sound waves from the subject 101 in real time, the control circuit 216 can objectively calculate physiological parameters using predetermined templates and/or functions. As such, the control circuit 216 can reduce dependence on the need to apply subjective knowledge in real time for a user to interpret the sound waves received by the microphone 208. The control circuit 216 can use the user interface 248 to present audio output data in combination with other sensor modalities. The user interface 348 can receive user input indicating instructions to zoom in, slow, speed up, or otherwise modify the output of the audio output data, and modify the output accordingly.

### Remote Medicine

The stethoscope system 200 can use one or both of the communications circuits 228, 240 to transmit information such as electronic audio signals, calculated physiological parameters, and/or calculated health conditions to remote devices. As such, the stethoscope system 200 can enable remote devices (e.g., user interfaces thereof) to present such information to remote users. In addition, the control circuit 216 can receive control instructions from remote devices via the communications circuits 228, 240, such as to control operation of the audio module 252 (e.g., to determine how to filter the signals outputted by the microphone 208).

### Therapy Evaluation

In some embodiments, the stethoscope system 200 can present information using the user interface 248 representative of how providing therapy to the subject 101 affects physiological parameters. For example, the control circuit 216 can use the microphone 208 to detect a pre-therapy electronic audio signal, and store the pre-therapy electronic audio signal in the database 264. A therapy may be provided to the subject 101. The control circuit 216 can receive an indication that the therapy is being provided to the subject 101, and detect a therapy electronic audio signal and store the therapy electronic audio signal in the audio database 264. The control circuit 216 can receive an indication that the therapy has been completed, and store a post-therapy electronic audio signal in the audio database 264. The control circuit 216 can output, using the user interface 248, at least two of the pre-therapy electronic audio signal, the therapy electronic audio signal, or the post-therapy electronic audio signal to enable a user to determine an effect of the therapy. The control circuit 216 can calculate comparisons amongst the pre-therapy, therapy, and post-therapy electronic audio signals. The control circuit 216 can similarly monitor and output indications regarding physiological parameters calculated based on the pre-therapy, therapy, and post-therapy electronic audio signals.

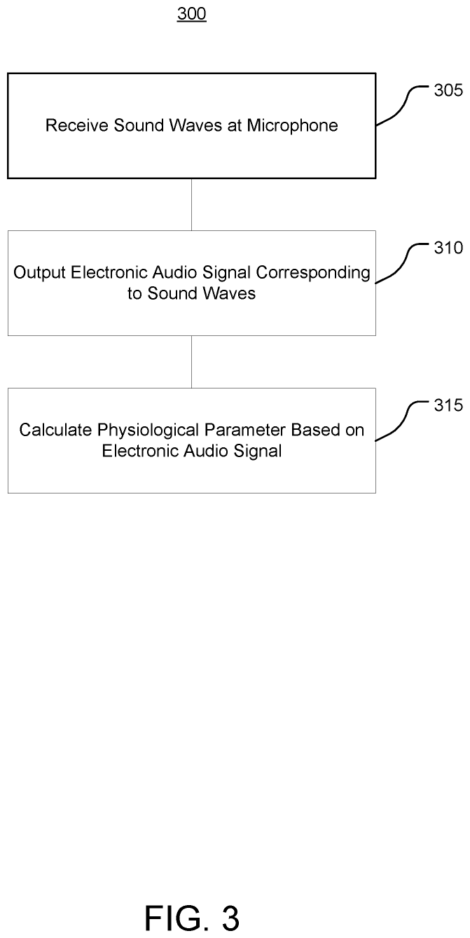

Referring now to FIG. 3, a method 300 of operating a stethoscope is shown according to an embodiment of the present disclosure. The method 300 can be performed by various systems and apparatuses described herein, including the stethoscope device 100 and the stethoscope system 200.

At 305, a plurality of sound waves are received from a subject by a microphone device. The microphone device may be provided in a stethoscope device, such as a handheld and/or portable device that can be placed in proximity to a particular region of the subject. At 310, the microphone device outputs an electronic audio signal corresponding to the plurality of sound waves.

At 315, a control circuit calculates a physiological parameter based on the audio signal. The physiological parameter can include various parameters, such as cardiac parameters, pulmonary parameters, fetal parameters, or gastrointestinal parameters. The control circuit can execute an audio filter on the electronic audio signal. The control circuit can select the audio filter based on a type of the physiological parameter. The control circuit can amplify or attenuate the audio signal (or portions thereof). The control circuit can determine a likelihood of the subject having a medical condition based on the physiological parameter.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the MIR and stethoscope devices and systems as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A stethoscope system, comprising:
a microphone device configured to receive a plurality of sound waves from a subject and output an audio signal corresponding to the plurality of sound waves; and
a control circuit configured to receive the audio signal from the microphone device and calculate a physiological parameter based on the audio signal.

2. The stethoscope system of claim 1, wherein the control circuit executes an audio filter on the audio signal prior to calculating the physiological parameter.

3. The stethoscope system of claim 2, wherein the control circuit selects the audio filter from a plurality of predetermined audio filters based on at least one of a physiological feature from which the plurality of sound waves were received or an expected type of the physiological parameter.

4. The stethoscope system of claim 1, wherein the control circuit includes a first processing circuit coupled to the microphone device by a wired connection, a first communications circuit coupled to the first processing circuit by a wired connection, a second processing circuit remote from the first processing circuit, and a second communications circuit configured to wirelessly receive data from the first processing circuit via the first communications circuit and provide the received data to the second processing circuit.

5. The stethoscope system of claim 1, wherein the control circuit includes a database mapping each calculated physiological parameter to at least one of a time of receipt of the corresponding plurality of sound waves, a location of receipt of the corresponding plurality of sound waves, or an identifier of the subject.

6. The stethoscope system of claim 1, wherein the microphone device is configured to receive the plurality of sound waves from at least one of a heart, a lung, an abdominal cavity, or a uterus of the subject.

7. The stethoscope system of claim 1, wherein the microphone device is configured to receive the plurality of sound waves from a vasculature of the subject, the vasculature including at least one of a neck vasculature or a leg vasculature.

8. The stethoscope system of claim 1, wherein the control circuit is configured to amplify at least a portion of the audio signal.

9. The stethoscope system of claim 1, wherein the control circuit is configured to output, using a display device, a visual representation of at least one of the audio signal or the physiological parameter.

10. The stethoscope system of claim 1, wherein the control circuit includes a parameter database storing a plurality of calculated physiological parameters.

11. The stethoscope system of claim 1, wherein the control circuit is configured to output the audio signal at a first rate less than a second rate at which the plurality of sound waves are received.

12. The stethoscope system of claim 1, wherein the control circuit is configured to estimate a physiological condition associated with the physiological parameter using a template of the physiological condition.

13. The stethoscope system of claim 1, wherein the control circuit is configured to cause a display remote from the microphone device to output a visual representation of the audio signal and modify the output of the visual representation based on a control signal received from a user interface coupled to the display device.

14. The stethoscope system of claim 1, wherein the control circuit maintains a database associating audio signal data to values of the physiological parameter, generates a function mapping audio signal data to values of the physiological parameter, and calculates the physiological parameter at least partially based on the function.

15. The stethoscope system of claim 1, wherein the control circuit is configured to overlay a first value of the calculated physiological parameter prior to delivery of therapy to the subject to a second value of the calculated physiological parameter.

16. The stethoscope system of claim 1, wherein the control circuit is configured to receive a request to provide output corresponding to a particular physiological parameter, retrieve, from a database, a plurality of electronic audio signals corresponding to the particular physiological parameter, and cause at least one of an audio output device to output at least a subset of the plurality of electronic audio signals or communications electronics to transmit the subset of the plurality of electronic audio signals.

17. The stethoscope system of claim 16, wherein the control circuit is configured to use the subset of the plurality of electronic audio signals to present a learning tool.

18. A method of operating a stethoscope, comprising:
receiving, by a microphone device, a plurality of sound waves from a subject;
outputting, by the microphone device, an audio signal corresponding to the plurality of sound waves; and
calculating, by a control circuit, a physiological parameter based on the audio signal.

19. The method of claim 18, comprising:
executing, by the control circuit, an audio filter on the audio signal prior to calculating the physiological parameter.

20. The method of claim 19, comprising:
selecting, by the control circuit, the audio filter from a plurality of predetermined audio filters based on at least one of a physiological feature from which the plurality of sound waves were received or an expected type of the physiological parameter.

21. The method of claim 18, comprising:
transmitting, from a first processing circuit of the control circuit to a second processing circuit of the control circuit, data regarding the audio signal, the first processing circuit coupled to the microphone device by a wired connection, the second processing circuit remote from the first processing circuit to wirelessly receive data from the first processing circuit.

22. The method of claim 18, comprising:
maintaining, by the control circuit, a database mapping each calculated physiological parameter to at least one of a time of receipt of the corresponding plurality of sound waves, a location of receipt of the corresponding plurality of sound waves, or an identifier of the subject.

23. The method of claim 18, comprising:
receiving, by the microphone device, the plurality of sound waves from at least one of a heart, a lung, an abdominal cavity, or a uterus of the subject.

24. The method of claim 18, comprising:
receiving, by the microphone device, the plurality of sound waves from a vasculature of the subject, the vasculature including at least one of a neck vasculature or a leg vasculature.

25. The method of claim 18, comprising:
amplifying, by the control circuit, at least a portion of the audio signal.

26. The method of claim 18, comprising:
outputting, by the control circuit using a display device, a visual representation of at least one of the audio signal or the physiological parameter.

27. The method of claim 18, comprising:
maintaining, by the control circuit, a parameter database storing a plurality of calculated physiological parameters.

28. The method of claim 18, comprising:
outputting, by the control circuit, the audio signal at a first rate less than a second rate at which the plurality of sound waves are received.

29. The method of claim 18, comprising:
estimating, by the control circuit, a physiological condition associated with the physiological parameter using a template of the physiological condition.

30. The method of claim 18, comprising:
causing, by the control circuit, a display remote from the microphone device to output a visual representation of the audio signal and modify the output of the visual representation based on a control signal received from a user interface coupled to the display device.

31. The method of claim 18, comprising:
maintaining, by the control circuit, a database associating audio signal data to values of the physiological parameter, generates a function mapping audio signal data to values of the physiological parameter, and calculates the physiological parameter at least partially based on the function.

32. The method of claim 31, comprising:
overlaying, by the control circuit, a first value of the calculated physiological parameter prior to delivery of therapy to the subject to a second value of the calculated physiological parameter.

33. The method of claim 18, further comprising:
receiving a request to provide output corresponding to a particular physiological parameter;
retrieving, from a database, a plurality of electronic audio signals corresponding to the particular physiological parameter; and
causing at least one of an audio output device to output at least a subset of the plurality of electronic audio signals or communications electronics to transmit the subset of the plurality of electronic audio signals.

34. The method of claim 33, further comprising using the subset of the plurality of electronic audio signals to present a learning tool.

35. A stethoscope system, comprising:
a microphone device configured to receive a plurality of sound waves from a subject and output an audio signal corresponding to the plurality of sound waves; and
a control circuit configured to receive the audio signal from the microphone device and maintain a record of the audio signal in memory.
